(19) United States
(12) Patent Application Publication
(10) Pub. No.: US 2014/0018621 A1
(43) Pub. Date: Jan. 16, 2014

(54) SYSTEMS FOR REDUCING FLUID LEAKAGE AND SPRAY-BACK FROM MEDICAL PROCEDURES

(71) Applicant: Christopher A. Stout, San Bruno, CA (US)

(72) Inventor: Christopher A. Stout, San Bruno, CA (US)

(21) Appl. No.: 13/550,416

(22) Filed: Jul. 16, 2012

(51) Int. Cl. A61B 1/00

(52) U.S. Cl. 600/106

(57) ABSTRACT

Assemblies and methods of inserting a delivery system assembly into a working channel are disclosed. In accordance with some embodiments, a sealing insert is configured to reduce fluid leakage and spray-back. In accordance with some embodiments, a sealing insert is disclosed in which a distal sealing surface is configured to seal against differently sized working channels of a plurality of endoscopes. In accordance with some embodiments, a sealing insert is disclosed in which a cap is configured to control a seal between a sealant and a delivery system assembly.

### BACKGROUND OF THE INVENTION

Embodiments of the present invention relate to the field of minimally invasive surgical medical devices and medical procedures. More specifically, embodiments of the present invention relate to devices and methods used for transcervical gynecological procedures.

Female contraception and sterilization may be enabled by transcervically introduced fallopian tube inserts. Devices, systems and methods for contraceptive approaches have been described in various patents and patent applications assigned to the present assignee. For example, U.S. Pat. No. 6,526,979, U.S. Pat. No. 6,634,361, U.S. patent application Ser. No. 11/165,733 published as U.S. Publication No. 2006/0293560 and U.S. patent application Ser. No. 12/605,304 describe transcervically introducing an insert (also referred to as implant and device) into an ostium of a fallopian tube and mechanically anchoring the insert within the fallopian tube. One example of such an assembly is known as “Essure”® from Conceptus, Inc. of Mountain View, Calif. Tissue in-growth into the “Essure”® insert induces long-term contraception and/or permanent sterilization.

An insert may be delivered into the fallopian tube with a delivery system assembly 100 such as the one illustrated in FIG. 1. The delivery system assembly 100 is formed of a control device 102 such as a handle, an elongated sheath 104, and an insert 106. The delivery system assembly may be transcervically introduced into the uterus and the fallopian tubes through a hysteroscope. Advancement of the delivery system assembly within the uterus and the fallopian tubes is usually facilitated by distending the uterus with a distention fluid, such as saline, and viewing the placement of the delivery system assembly through the hysteroscope.

Referring to FIG. 2 the hysteroscope 200 may include a nozzle 204 including a valve clamp 208, such as a ball valve clamp, and an access port 206 positioned at a proximal end of the nozzle 204 to access a working channel 202 into which the delivery system assembly 100 is inserted. Closing the valve clamp 208 may seal the entrance of the working channel 202 to prevent distention fluid from leaking out of the access port 206 when a delivery system assembly 100 does not occupy the working channel 202 of the hysteroscope 200. A sealing cap 230 including a proximal seal 232, can be fitted over the outer diameter of nozzle 204 containing the access port 206 to prevent distention fluid from leaking out of the hysteroscope 200 when a delivery system assembly 100 occupies the working channel of the hysteroscope system.

An introducer 220 may be used in order to prevent damaging the tip of the elongated sheath 104 or insert 106 of the delivery system assembly 100 during insertion through the proximal seal 232 of the sealing cap 230 and access port 206, and into the working channel 202 of the hysteroscope system 200. Introducer 220 includes a sheath portion 222 and slit opening 224 to aid in grasping and in the removal of the introducer 220. The introducer 220 is inserted through the proximal seal 232 of the sealing cap 230 and into the working channel 202 prior to inserting the delivery system assembly 100. When the introducer 220 is inserted through the sealing cap 230, fluid can spray out of the introducer 220 and onto the physician or physician's assistant. This result, i.e., the spray of fluid out of sealing cap 230 or the introducer 220 is commonly referred to as “spray-back”. The amount of fluid spray-back can be significant depending on the pressure of the distention fluid used during the procedure.

Referring to FIG. 3, after placing the introducer 220 into the working channel 202, the tip of delivery system assembly 100 is inserted into the slit opening 224 and through the sheath 222 of the introducer 220 in order to advance the delivery system assembly 100 into the working channel 202 of the hysteroscope. This is typically performed as soon as possible after placement of the introducer 220 into the working channel 202 in order to minimize the amount of fluid spray-back from the introducer. The introducer 220 may then be removed or may be kept in place throughout the procedure. After insertion of the delivery system assembly 100 into the introducer 220, an amount of distention fluid may still leak from between the introducer 220 and elongated sheath 104 of the delivery system assembly 100, as well as from the between sealing cap 230 and nozzle 204.

### SUMMARY OF THE DESCRIPTION

Embodiments of the present invention generally provide assemblies to facilitate the insertion of a delivery system assembly into a working channel of an endoscopic system, such as the insertion of a delivery system assembly into a hysteroscope for accessing a female reproductive system. While embodiments of the invention are described with reference to a hysteroscope, it is to be understood that the embodiments are not limited to such and may also be compatible with other optical surgical devices and endoscopy systems. It is to be further understood that the embodiments may be compatible with other systems used to access the human body, such as guiding catheters, by way of example. In one aspect, embodiments of the invention describe systems which may reduce the amount of fluid spray-back and leakage associated with inserting a delivery catheter into a working channel of a hysteroscope. In another aspect, embodiments of the invention describe a sealing insert that can prevent leakage and spray-back when the sealing insert is fit into a nozzle containing an access port to the working channel of the hysteroscope. Such prevention can be provided whether or not a delivery system assembly is inserted into the sealing insert. In another aspect, the sealing insert can be compatible with a plurality of commercially available hysteroscopes having nozzles of different dimensions.

One embodiment of the present invention relates to a sealing insert which is configured to prevent or reduce fluid leakage and spray-back. The sealing insert can include a sealant that has an aperture. The sealing insert can also include means for sealing the sealing insert against differently sized ports and/or working channels of a plurality of endoscopes. For example, in one embodiment, the means can be a distal sealing surface of the sealing insert. Furthermore, the sealing insert can include means for actuating the aperture to control a seal of the sealant. For example, in one embodiment, the means can be a movable cap that actuates the aperture while a delivery system assembly is positioned within the sealant to effect a seal between the sealant and the delivery system assembly.

In another embodiment, a sealing insert can include a housing with a distal sealing surface configured to seal against differently sized working channels of a plurality of endoscopes. The housing can also include a lumen and a seat, and the housing can be coupled with a cap having a port. The sealing insert can also include a sealant having an aperture, the sealant being disposed between the seat and the cap such that the aperture, the lumen, and the port align to allow a delivery system assembly to be introduced through the port, aperture, and lumen into one of the differently sized working channels of the plurality of endoscopes. Movement of the cap relative to the housing can actuate the aperture to control a seal between the sealant and the delivery system assembly.

In another embodiment, a sealing insert can include a housing, a cap, and a sealant as previously described. Furthermore, the sealing insert can include an introducer disposed through the port, the introducer having a protuberance and a passage. The protuberance can be positioned between the sealant and the cap. Movement of the cap can bias the protuberance to actuate the introducer to advance into, or retract from, the aperture. The passage can be configured to introduce the delivery system assembly through the port, the aperture, and the lumen into one of the differently sized working channels of the plurality of endoscopes.

Another embodiment of the present invention relates to a kit which may include a delivery system assembly and a sealing insert. The delivery system assembly can include a control device, an elongated catheter sheath having a proximal end connected to the control device, and an insert. The sealing insert can have a housing, a cap, and a sealant as previously described. The sealing insert can additionally be configured to accommodate a plurality of commercially available endoscopes having nozzles of different outside dimensions as previously described.

The above summary does not include an exhaustive list of all aspects of the present invention. It is contemplated that the invention includes all systems and methods that can be practiced from all suitable combinations of the various aspects summarized above, and also those disclosed in the Detailed Description below.

### BRIEF DESCRIPTION OF THE DRAWINGS

The present invention is illustrated by way of example and not limitation in the figures of the accompanying drawings in which like references indicate similar, but not necessarily identical, elements.

FIG. 1 is a cross-sectional view illustration of a delivery system assembly.

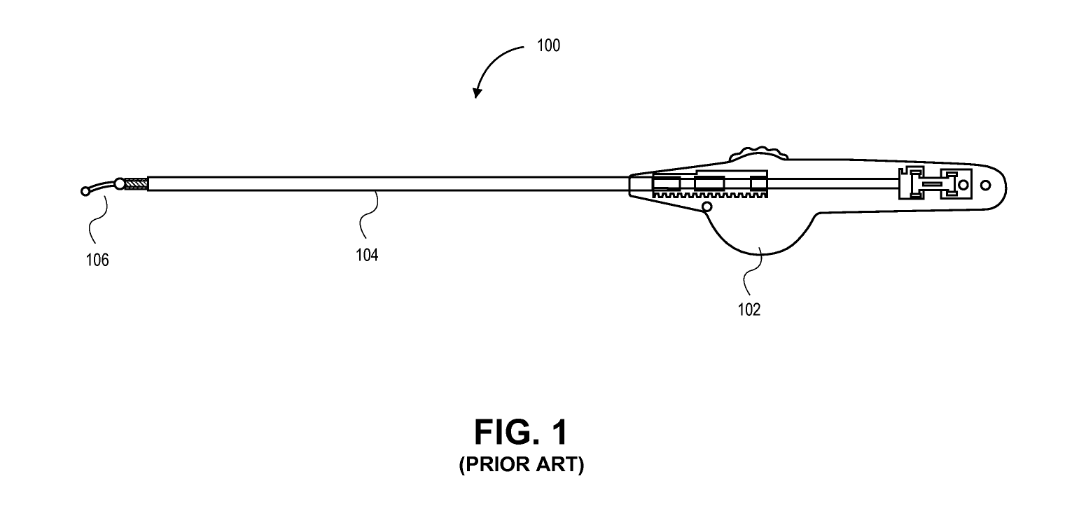

FIG. 2 is a perspective view illustration of a hysteroscope and an introducer.

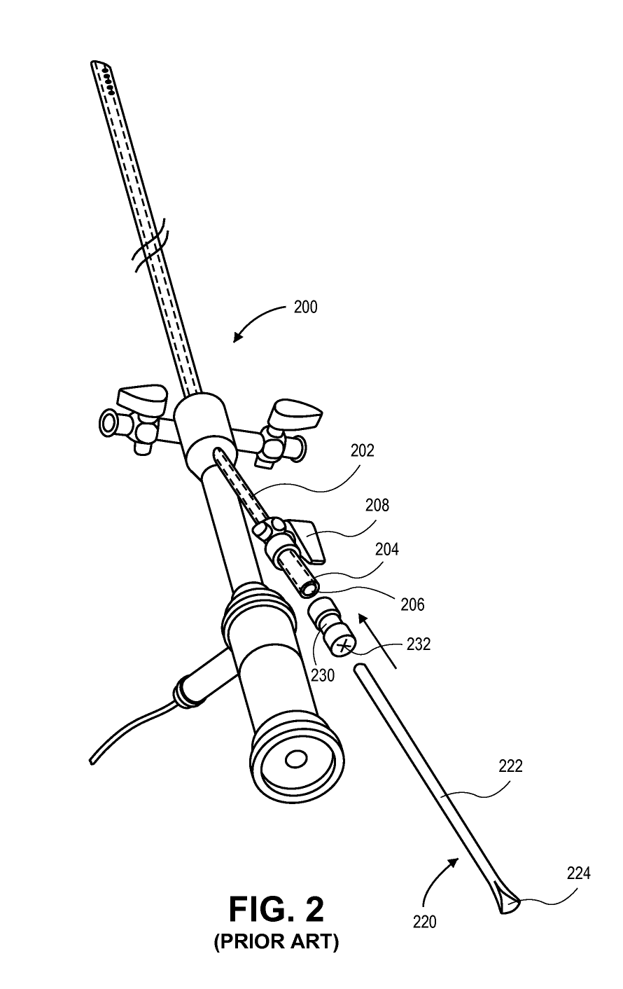

FIG. 3 is a perspective view illustration of a delivery system assembly inserted into an introducer and working channel of a hysteroscope.

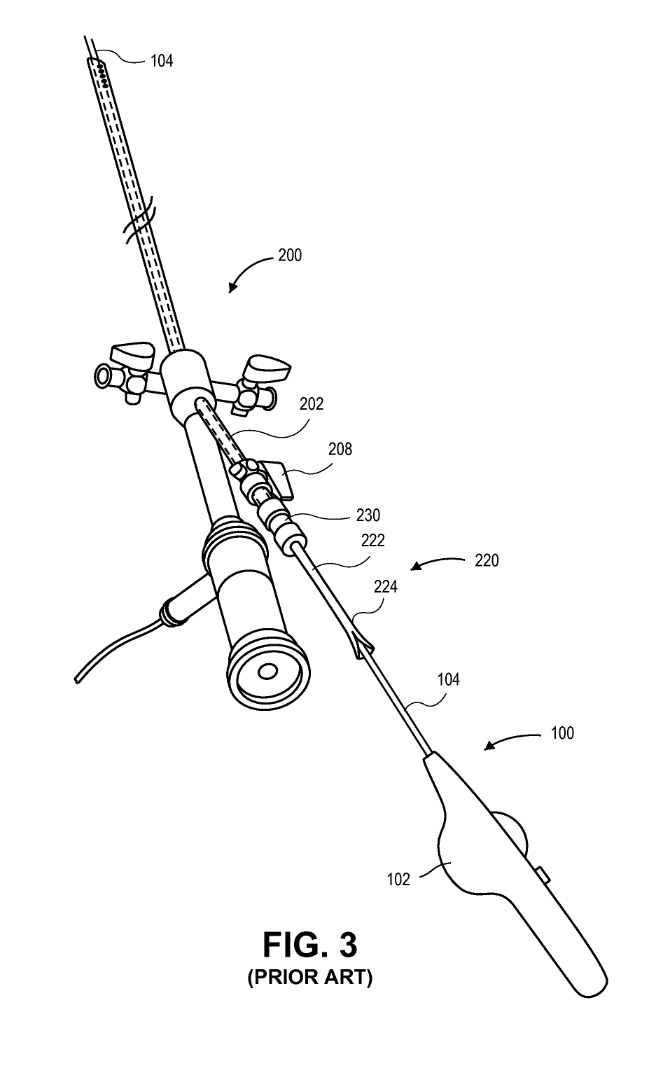

FIG. 4 is a cross-sectional view illustration of a sealing insert in accordance with an embodiment of the invention.

FIGS. 5A-5D are cross-sectional view illustrations of a sealing insert in accordance with an embodiment of the invention.

FIG. 6 is a cross-sectional view illustration of a sealing insert in accordance with an embodiment of the invention.

FIG. 7 is a cross-sectional view illustration of a sealing insert in accordance with an embodiment of the invention.

FIG. 8 is a perspective view illustration of a sealant component of a sealing insert in accordance with an embodiment of the invention.

FIG. 9 is a cross-sectional view illustration, taken about section line A-A of FIG. 8, of a sealant component of a sealing insert in accordance with an embodiment of the invention.

FIG. 10 is a perspective view illustration of a sealant component of a sealing insert in accordance with an embodiment of the invention.

FIG. 11 is a cross-sectional view illustration, taken about section line A′-A′ of FIG. 10, of a sealant component of a sealing insert in accordance with an embodiment of the invention.

FIGS. 12A-12C are isometric view illustrations of inserting a delivery system assembly into a working channel of a hysteroscope system in accordance with an embodiment of the invention.

### DETAILED DESCRIPTION

Various embodiments and aspects of the inventions will be described with reference to details discussed below, and the accompanying drawings will illustrate the various embodiments. The following description and drawings are illustrative of the invention and are not to be construed as limiting the invention. Numerous specific details are described to provide a thorough understanding of various embodiments of the present invention. However, in certain instances, well-known or conventional details are not described in order to provide a concise discussion of embodiments of the present invention.

Reference in the specification to “one embodiment” or “an embodiment” means that a particular feature, structure, or characteristic described in conjunction with the embodiment can be included in at least one embodiment of the invention. The appearances of the phrase “in one embodiment” in various places in the specification do not necessarily all refer to the same embodiment. Although the processes are described below in terms of some sequential operations, it should be appreciated that some of the operations described may be performed in a different order. Moreover, some operations may be performed simultaneously rather than sequentially.

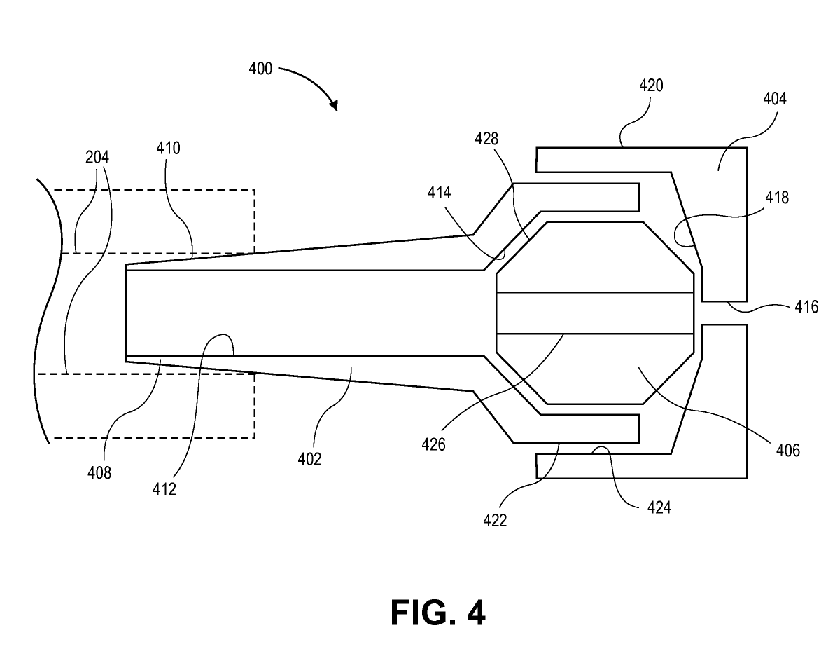

Referring to FIG. 4, a full sectional projected view illustration of a sealing insert 400 in accordance with an embodiment of the invention is shown. In this embodiment, the sealing insert 400 includes a housing 402, a cap 404, and a sealant 406. The configuration of these components, and of the sealing insert as a whole, permit sealing between the sealing insert and a working channel of an endoscopic system and/or a surface of a delivery system assembly to prevent or reduce fluid leakage and spray back from a medical procedure. For instance, the sealing insert 400 can seal against a working channel 202 of a hysteroscope 200 used for accessing a female reproductive system. Further, the sealing insert 400 can seal against an outer surface of a delivery system assembly 100 used to deliver a contraceptive insert into a female reproductive system through the hysteroscope.

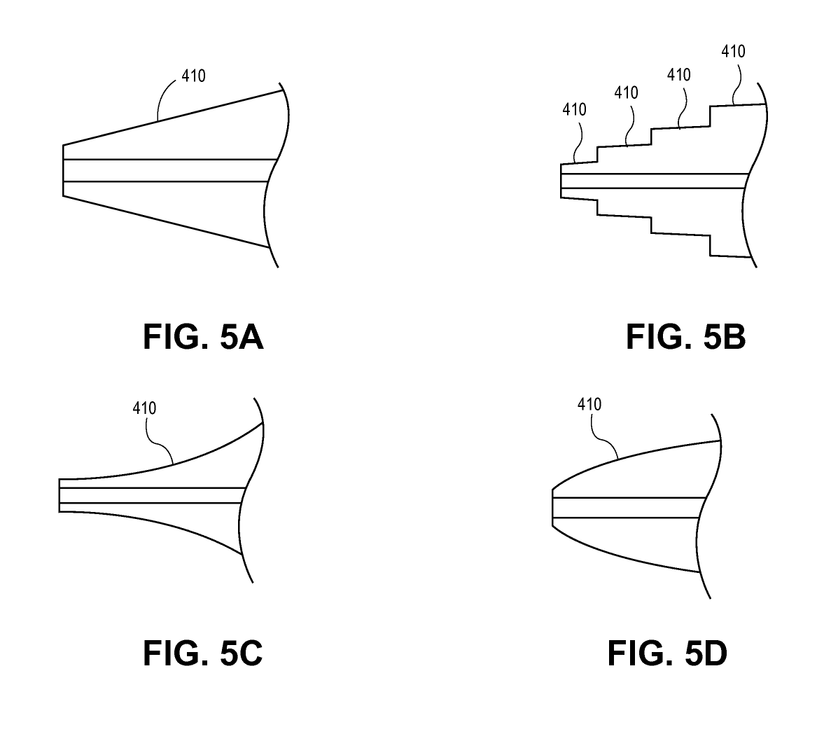

In one embodiment, the housing 402 includes a distal nipple 408 having a distal sealing surface 410 that can be inserted into a nozzle, or port, of a hysteroscope working channel. The working channel can be used, for example, to pass a delivery system assembly into a patient. The distal sealing surface 410 can be configured to engage with a surface of the port or working channel. In one embodiment, the distal sealing surface can be configured to fit within the inner diameter of the port or working channel of the hysteroscope, such as the inner diameter of nozzle 204, and FIG. 4 shows an example of this embodiment. Furthermore, the distal sealing surface can be configured to accommodate, e.g., seal against, ports or working channels of various sizes. This flexibility can be achieved by providing a distal sealing surface 410 having a varying profile. For example, the profile of the distal sealing surface can include an outer dimension that varies over a length. In one embodiment, the outer dimension can be an outer diameter that reduces or increases in size in at least one direction, e.g., the profile of the distal sealing surface 410 can taper in a distal direction toward a tip of the distal nipple 408. Various alternative embodiments of the distal sealing surface 410 are provided below in reference to FIGS. 5A-5D.

Referring to FIG. 5A, a partial section projected view illustration of a sealing insert in accordance with an embodiment of the invention is shown. In this embodiment, the sealing insert includes a distal sealing surface 410 having a profile that is tapered over at least a portion of its length. The taper can be continuous, and it can have a tapering ratio defined by a taper rise over a taper run. The tapering ratio can be chosen to facilitate an effective seal between a working channel and the distal sealing surface when the sealing insert is placed within a port or working channel. Only by way of example, the tapering ratio could be approximately 1:10, corresponding to a taper angle of approximately five degrees. However, it will be appreciated that an effective tapering ratio to achieve a seal between the distal sealing surface and a working channel can depend on various factors, e.g., the material used to form the housing 402 and/or distal nipple 408.

Referring to FIG. 5B, a partial section projected view illustration of a sealing insert in accordance with an embodiment of the invention is shown. In this embodiment, the sealing insert includes a distal sealing surface 410 having a profile that is stepped over a portion of its length. The outer dimension of each progressive step can be chosen to engage with a working channel of a different hysteroscope. For example the outer dimension of the distal step can be chosen to fit a working channel in an endoscope manufactured by one manufacturer, while each subsequent stepped outer dimension can be sized to engage the working channel of endoscopes manufactured by another manufacturer. It will also be appreciated that each stepped portion of the stepped distal sealing surface 410 can be straight or tapered. Thus, the stepped configuration may allow a distal sealing surface having a shorter length to accommodate a wider range of working channel diameters.

Referring to FIG. 5C, a partial section projected view illustration of a sealing insert in accordance with an embodiment of the invention is shown. In this embodiment, the sealing insert includes a distal sealing surface 410 having a concave profile. It will be appreciated that the concave profile allows for the distal sealing surface to engage a variety of working channels. Furthermore, the concave configuration may allow for a distal sealing surface having a shorter length to accommodate a wider range of working channel.

Referring to FIG. 5D, a partial section projected view illustration of a sealing insert in accordance with an embodiment of the invention is shown. In this embodiment, the sealing insert includes a distal sealing surface 410 having a convex profile. It will be appreciated that the convex profile allows for the distal sealing surface to engage a variety of working channels. Furthermore, the convex configuration may allow for a distal sealing surface having a shorter length to accommodate a wider range of working channel diameters.

In alternative embodiments, the distal sealing surface 410 may be shaped in a manner that permits the sealing insert to fulfill the function of sealing against a port or a working channel of an endoscopic system. By way of example and not limitation, such shape can be frustoconical, zig-zagged, undulating, and so forth.

Strictly by way of example, the distal sealing surface 410 can be designed to accommodate ports or working channels having diameters in the range of about 0.05-inches to about 0.2-inch. Such a design would allow the distal sealing surface to seal against working channels having dimensions of 0.098-inch, 0.099-inch, 0.123-inch, 0.126-inch, and 0.153-inch. Those dimensions, although only illustrative, correlate to the dimensions of working channels in several currently available endoscopic systems. For example, an embodiment of a sealing insert 400 having a stepped distal sealing surface 410 can include a distal sealing surface having steps measuring approximately 0.097-inch, 0.100-inch, 0.127-inch, 0.128-inch, 0.151-inch, and 0.154-inch in diameter. Again, these dimensions are provided solely for the purpose of illustration and are not to be construed as a limitation of the range of ports or working channels that a sealing insert in accordance with this description may accommodate.

It will be appreciated that although the distal sealing surface 410 has been referred to above as being a feature or a portion of housing 402, the distal sealing surface 410 can in fact be part of a separate component. For example, the distal sealing surface 410 can be a feature of a nipple 408 or a nipple sleeve (not shown) that is sized and configured to couple with the housing 402. In one embodiment, the distal sealing surface 410 can be a conical sleeve that is placed over a distal portion of the housing 402. Thus, the distal sealing surface can be separated from the housing. It will be appreciated that such an embodiment would allow for the distal sealing surface, or the component that includes the distal sealing surface, to be formed from a different material than that used to form the housing. Therefore, a more resilient or tear resistant material can be used to form the distal sealing surface and a more rigid material can be used to form the housing. Advantageously, this difference in material could allow for a more a robust seal to be formed between the distal sealing surface and the port or working channel than may otherwise be formed if the distal sealing surface is fabricated from the same material as the housing.

By way of example and not restriction, several advantages are provided by a sealing insert 400 having a distal sealing surface 410 such as the one described in the embodiments above and encompassed within the scope of this description. First, the distal sealing surface can fit within ports and working channels of various sizes while ensuring a secure seal against these ports and working channels. Second, the fit between the distal sealing surface and the various port and working channels is more repeatable, due to the distal sealing surface being able to accommodate all diameters over a range. Thus, manufacturing tolerances of the sealing insert and the endoscopic systems may be less stringent. Yet another advantage of some sealing insert embodiments as compared to known distension valves is that the insertion of the distal sealing surface within a nozzle, rather than around a nozzle, reduces the likelihood that a nozzle may inadvertently puncture or damage an internal component of the sealing insert.

Referring again to FIG. 4, the housing 402 can also include a lumen 412 formed through at least a portion of the housing length. The lumen 412 can facilitate the passage of both instruments and fluids used during an endoscopic procedure. For example, the lumen 412 can be sized to allow for the passage of a delivery system assembly 100. The lumen 412 can also be sized to permit adequate flow in case the sealing insert 400 is to be used for insertion or extraction of fluids during an endoscopic procedure. Although the lumen 412 can have a smooth surface in one embodiment, the lumen can be textured, shaped, or surface treated to promote fluid and device passage in another embodiment.

In one embodiment, the housing 402 includes a seat 414 feature. The seat can be embodied as a recess within the housing body. The recess can include a surface to receive a sealant 406. As such, the seat 414 can be sloped, or otherwise shaped, to prevent the sealant 406 from being flushed through the housing lumen 412. Furthermore, the seat 414 shape can facilitate an appropriate seal with the sealant 406 by conforming to the sealant shape or by allowing the sealant to conform and create a seal against the seat. For example, the seat 414 can include various ridges or other features that grip and seal against the sealant 406 when pressure is applied between the seat and sealant, thereby resisting spray-back or flow of fluids between the seat and the sealant. Alternatively, the seat 414 can be embodied as a chamfer or fillet feature in the recess.

The housing 402 can be formed from any suitable material known in the art that possesses sufficient material properties to enable the functions described throughout this description. For example, the housing 402 can be formed from a material sufficiently rigid to permit the housing to compress and seal against the sealant. Furthermore, it may be important for the housing to possess adequate biocompatibility. By way of example, and not limitation, the housing can be fabricated from medical grade plastics such as polypropylene, polyamide, or other suitable materials. Various manufacturing processes can be used to form the housing, including injection molding and machining.

Referring still to FIG. 4, in one embodiment, the cap 404 can include a port 416 formed through at least a portion of its length. The port 416 can facilitate the passage of both instruments and fluids used during an endoscopic procedure. Therefore, the port 416 can be sized to allow for the passage of a delivery system assembly 100. The port can also be sized to permit adequate flow in case the sealing insert is to be used for insertion or extraction of fluids during an endoscopic procedure.

In some embodiments, the port 416 can include features to ease the insertion of a delivery system assembly through the port. For example, the port can include a chamfer, fillet, or other types of lead-in feature on one or both ends to ease insertion and removal of a delivery system assembly and to prevent snagging of components, such as contraceptive inserts, that are delivered through the sealing insert 400.

The cap 404 can also include a cap recess 418 feature. The cap recess 418 can be embodied as a recess within the cap body, wherein the recess includes a surface to engage with a sealant 406. As such, the cap recess 418 can be sloped, or otherwise shaped, to prevent the sealant 406 from being flushed out of the cap 404. Furthermore, the cap recess 418 can facilitate an appropriate seal with the sealant 406 by pressing the sealant against the housing seat 414. Thus, in one embodiment, the sealant 406 is configured to be constrained between the housing seat 414 and the cap recess 418. As such, the cap recess 418 can include features similar to those described above with respect to the housing seat 414, which grip and seal against the sealant 406.

In one embodiment, the cap 404 can also include features that allow the cap 404 to be easily gripped and moved. For example, the cap grip 420 can be roughened, knurled, embossed, patterned, or otherwise modified to provide a surface that is more easily gripped by a user. In another embodiment, the cap 404 can instead include a cap grip 420 with a substantially smooth surface.

The cap 404 can be formed from any suitable material known in the art possessing sufficient material properties to enable the functions described throughout this description. For example, the cap 404 can be formed from a material that is sufficiently rigid to permit the cap 404 to compress the sealant 406. Furthermore, it may be important for the cap 404 to possess adequate biocompatibility. By way of example, and not limitation, the cap can be fabricated from medical grade plastics such as polypropylene, polyamide, or other suitable materials. Various manufacturing processes can be used to form the cap, including injection molding and machining.

In one embodiment, the housing 402 and the cap 404 are coupled through a housing fastener 422 and a cap fastener 424. It will be appreciated that the coupling can be provided in a manner that allows the housing 402 and cap 404 to either be fixed or to move relative to each other. Furthermore, movement between the housing 402 and the cap 404 can be in one or more directions. For example, the coupling can allow and/or enable the housing 402 and the cap 404 to be moved axially or rotationally relative to each other.

In one embodiment, the cap 404 and the housing 402 can be coupled through a threaded fastener. For example, the housing fastener 422 and cap fastener 424 can both be threaded to allow the cap 404 and housing 402 to be rotated relative to each other. It will be appreciated that this coupling would permit the housing 402 and cap 404 to be moved both rotationally and axially relative to each other.

In an alternative embodiment, the cap 404 and the housing 402 can be coupled using a mechanical linkage. For example, the housing fastener 422 could include a key feature that engages a slot formed in the cap fastener 424. The slot could be spiral, axial, or otherwise directed in order to allow the housing 402 to be moved relative to the cap 404 by advancing the key feature through the slot. Thus, such a coupling would permit the housing 402 and cap 404 to be moved relative to each other in a manner defined by the slot path. For example, if the slot is axially directed, then movement of the protrusion in the slot would produce an axial movement of the housing 402 relative to the cap 404.

It will be appreciated that various other coupling designs can be used that vary, or are in addition to, the couplings discussed above. Any of these designs may facilitate movement between the housing 402 and cap 404 to affect a sealant 406 disposed between the housing 402 and cap 404. As will be described further below, the effect on the sealant 406 can include actuating an aperture of the sealant 406 to control a seal provided by the sealant 406.

Referring still to FIG. 4, in one embodiment, the sealant 406 can include an aperture 426 formed through at least a portion of the sealant length. The aperture 426 can facilitate the passage of both instruments and fluids used during an endoscopic procedure. Therefore, the aperture 426 can be sized to allow for the passage of a delivery system assembly 100. The aperture 426 can also be sized to permit adequate flow in case the sealing insert 400 is also to be used for insertion or extraction of fluids used during an endoscopic procedure. Furthermore, the aperture 426 can be configured to form a seal against a surface of a delivery system assembly 100 that is passed through the aperture 426.

The sealant 406 can include an outer shape 428 that conforms to a portion of the housing seat 414 and the cap recess 418. For example, the outer shape 428 can be sloped, curved, undulating, or any other shape that enables the function of sealing against a surface of the seat 414 or cap recess 418. In an embodiment, it is beneficial for the sealant 406 to include an outer profile having an outer dimension that varies, such that an outer dimension of the sealant 406 near an opening to the aperture 426 is less than an outer dimension of the sealant 406 near a medial portion of the aperture 426. It will be appreciated that such a shape can allow for axial compressive forces to be radially directed through the sealant 406 to reduce the aperture size.

The sealant 406 can be formed from any suitable material known in the art that possesses sufficient flexibility, strength, and tear resistance to permit the sealant 406 to be resiliently compressed, to form and release a seal, around a delivery system assembly 100. Thus, suitable materials can include silicones, fluoroelastomers, or other rubbers or elastomeric materials. However, it will be appreciated that suitable materials can also include any other rigid, semi-rigid, or non-rigid material that permits a seal to be formed between the sealant 406 and a delivery system assembly 100.

A seal between the sealant 406 and a delivery system assembly 100 can be facilitated by increasing and decreasing the aperture size. In one embodiment, the size of the aperture 426 can depend on the compressive loads applied to the outer surface of the sealant 406. For example, as shown in FIG. 4, the sealant 406 can have an annular shape about the aperture 426. Compressive loads can be placed on the sealant 406 by the seat 414 and/or cap recess 418 to generate sufficient stresses in the sealant 406 to cause the sealant to deform inwardly. This deformation can cinch and thereby reduce the size of the aperture 426. The size reduction can eventually close the aperture 426 entirely in at least one location, thereby sealing the sealant 406 against the flow of fluid. Alternatively, the size reduction can close the sealant 406 around a delivery system assembly 100 disposed through the aperture 426, thereby preventing the flow of fluid between the delivery system assembly 100 and sealant 406.

Thus, at least one embodiment of a sealing insert 400 is provided that can be inserted within a working channel of a hysteroscope to form a seal to prevent fluid leakage. Further, sealing insert 400 includes an aperture that can be actuated to control a seal between a sealant and a delivery system assembly to prevent fluid spray-back. In one embodiment, the aperture can be actuated by moving the cap 404 towards sealant 406 to cause the sealant to deform inwardly and to thereby reduce a cross-sectional size of aperture 426.

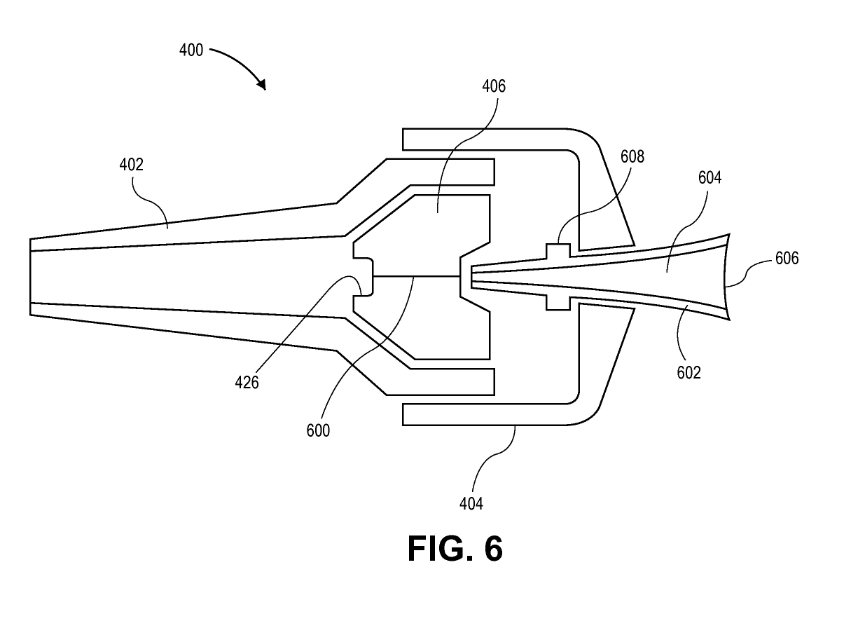

Referring now to FIG. 6, a full sectional projected view illustration of a sealing insert 400 in accordance with an alternative embodiment of the invention is shown. This embodiment of a sealing insert varies with respect to the embodiment discussed above in at least two aspects. First, the sealing insert can include a self-closing sealant 406. Second, the sealing insert can further include an introducer 602 component.

In an embodiment, the self-closing sealant 406 can be configured to prevent fluid leakage and spray-back when in a closed state, while allowing the sealant 406 to be pierced or opened by the advancement of an object. For example, in certain embodiments, the self-closing sealant 406 can be designed as a slit seal through which an introducer 602, guidewire, and/or delivery system assembly can be advanced. The slit seal can be formed, for example, by slitting a membrane of the sealant 406 with a sharpened object in order to create a slit 600 therethrough.

It will be appreciated that the self-closing seal can be embodied by any number of other seals and valves that fulfill the self-closing purpose while also permitting the passage of an object. As an example, the self-closing seal can be embodied as a duckbill valve. One skilled in the art will appreciate that the valve design choice can depend on certain considerations, such as the required crack pressure or flow characteristics of the valve.

As such, the self-closing sealant 406 can be formed from any suitable material known in the art that possesses sufficient flexibility, strength, and tear resistance to allow for the intermittent opening and self-closing that it may undergo during use. For example, suitable materials can include silicones, fluoroelastomers, or other rubbers or elastomeric materials.

Referring still to FIG. 6, an embodiment of the sealing insert 400 can include an introducer 602. The introducer 602 can be configured to protect the distal end of an object, such as the distal tip of a delivery system assembly, during advancement of the object through the cap 404, sealant 406, and housing 402. That is, the introducer 602 can be configured to prevent damage to, and ease the insertion of a delivery system assembly as it is advanced through aperture 426 of the sealing insert into a working channel of an endoscopic system.

In an embodiment, the introducer 602 can comprise a passage 604 disposed through the introducer length and axially aligned with an aperture 426 and/or slit 600 of the sealant 406. The passage 604 can be configured to ease insertion of, e.g., a delivery system assembly 100. For example, the passage 604 can be flared near an entry 606 such that the distal tip of a delivery system can be more easily inserted into the entry 606 and be guided toward the aperture 426.

Furthermore, the introducer 602 can include a protuberant feature 608 that extends outward from an outer surface of the introducer 602. The protuberant feature can be sized and shaped to be retained between the cap 404 and the sealant 406, while resisting movement through either. For example, in one embodiment, the protuberant feature could be a radially formed flange having an outer dimension that is greater than the greatest outer diameter of both the cap port 416 and the sealant aperture 426. In an alternative embodiment, the protuberance 608 can be a bulge formed either separately or integrally with the introducer body. The bulge can have an outer dimension that is greater than the greatest outer diameter of both the cap port 416 and the sealant aperture 426. The bulge can be molded or overmolded onto the introducer body, or it could be formed by an adhesive bead added to the introducer surface after formation, for example. Further still, the protuberance 608 could be a separate component, such as an o-ring, that is coupled with the surface of the introducer. Thus, it will be appreciated that the protuberance 608 can be formed in many ways known in the art. In any case, the protuberance 608 can keep the introducer 602 retained within the sealing insert 400 assembly while enabling some degree of movement of the introducer therein. For example, the introducer may be advanced and retracted between locations where the protuberance 608 contacts the cap 404 and sealant 406. In one embodiment, when the introducer is retracted such that protuberance 608 contacts cap 404, the aperture 426 can be in a closed configuration. However, when the introducer is advanced such that protuberance 608 contacts sealant 406, the aperture can be in an open configuration.

The introducer 602 can be formed from any suitable material known in the art that possesses sufficient flexibility, strength, and surface characteristics to facilitate the introduction of delivery system assemblies therethrough. For example, suitable materials can include polyamides, polyimides, polytetrafluoroethylene, or other suitable materials. An example of an introducer that incorporates a suitable material is the DryFlow™ introducer available from Conceptus, Inc. of Mountain View, Calif.

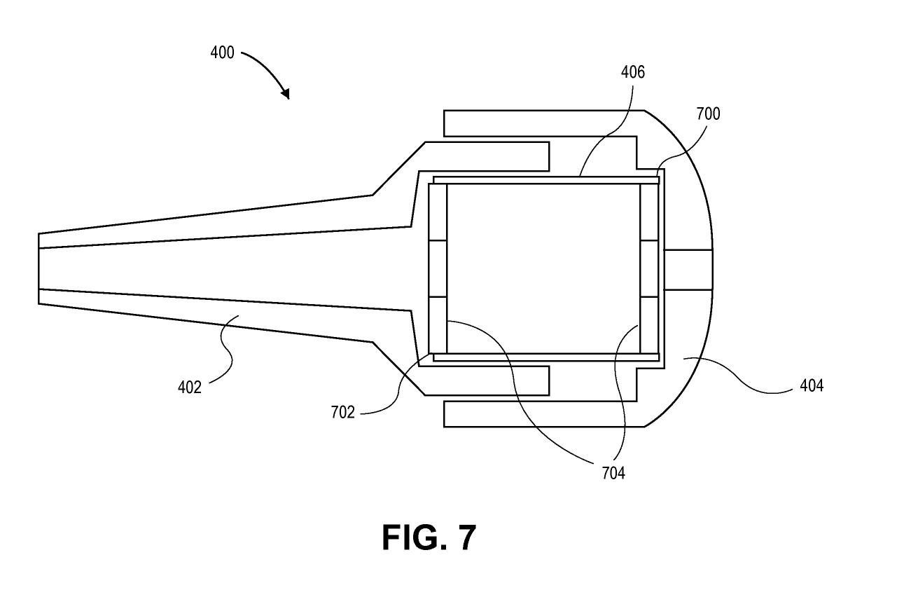

Referring to FIG. 7, a full sectional projected view illustration of a sealing insert 400 in accordance with an embodiment of the invention is shown. The sealing insert 400 includes an alternative embodiment of the sealant 406. The sealant 406 includes a first end 700 and a second end 702. The first end 700 and the second end 702 can be coupled to mounting rings 704, or alternatively, the first end 700 and the second end 702 can be coupled to the cap 404 and the housing 402. In the case of the first end 700 and the second end 702 being coupled to the mounting rings 704, the mounting rings can further be coupled to, or positionally associated with, the cap 404 and the housing 402. Thus, movement of the cap 404 relative to the housing 402 can produce a similar relative movement between the first end 700 and the second end 702 of the sealant 406. The coupling between the mounting rings and cap or housing can be fixed, e.g., by an adhesive bond. Alternatively, the coupling can be temporary or transient, as characterized by a friction coupling of the mounting rings to the cap or housing.

The mounting rings 704 can further include a port or passage that can be aligned with the port 416 of the cap 404 and the lumen 412 of the housing 402. Thus, a delivery system assembly 100 can pass freely through the sealing insert 400 when the sealant 406 is in an open configuration. However, the sealant can be closed to prevent the advancement of a delivery system assembly and/or reduce spray-back by sealing against itself or against a delivery system assembly positioned therethrough.

The sealant 406 can be coupled to the mounting rings 704, or to the cap 404 and housing 402, through a variety of manufacturing techniques. For example, the sealant can be bonded to the mounting rings by an adhesive. Alternatively, the mounting rings and the sealant can be bonded by a thermal or mechanical weld. Further still, the mounting rings can be inserted or press fit into the aperture 426 of the sealant 406.

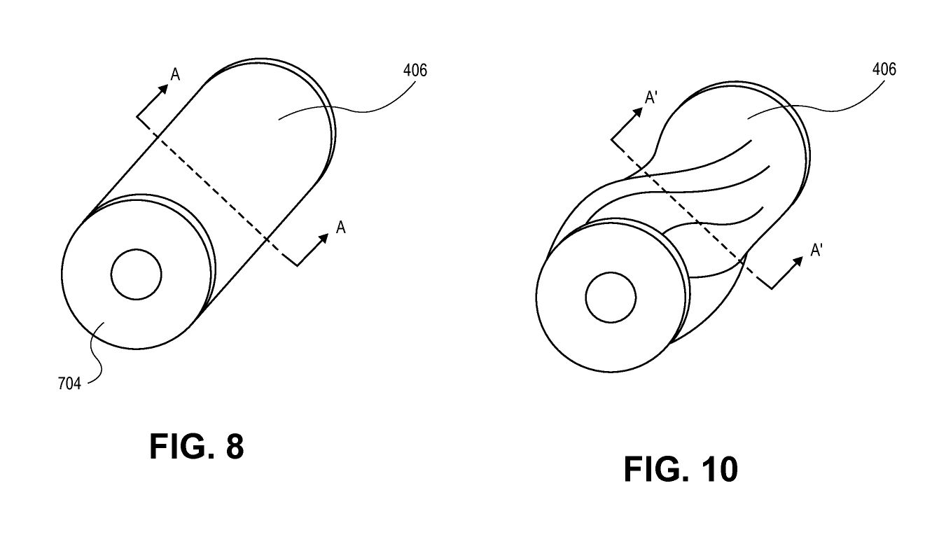

Referring to FIG. 8, a perspective view illustration of a sealant 406 component of a sealing insert in accordance with an embodiment of the invention is shown. In this embodiment, the sealant 406 is shown in an open configuration, in which the sealant body has a tubular configuration. Such a configuration could, for example, have an outer diameter and an inner diameter that remain substantially continuous over the length of the sealant. Alternatively, the tubular body can have a varying profile. For example, the inner diameter could be shaped in a convex manner such that the inner diameter is less near a medial location of the sealant body than near the first end 700 and the second end 702 of the sealant. Numerous alternative profiles can be used as well, as will be appreciated by one skilled in the art.

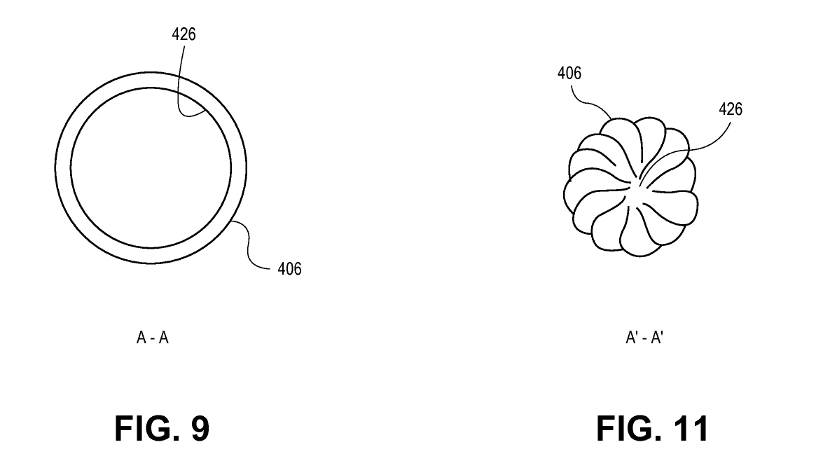

Referring to FIG. 9, a full sectional projected view illustration, taken about section line A-A of FIG. 8, of a sealant 406 component of a sealing insert in accordance with an embodiment of the invention is provided. As shown, while in the open configuration, a cross-section of the sealant 406 can include an aperture 426 that is substantially circular. Thus, a delivery system assembly and/or fluid can move or flow freely through the aperture 426 and the sealing insert.

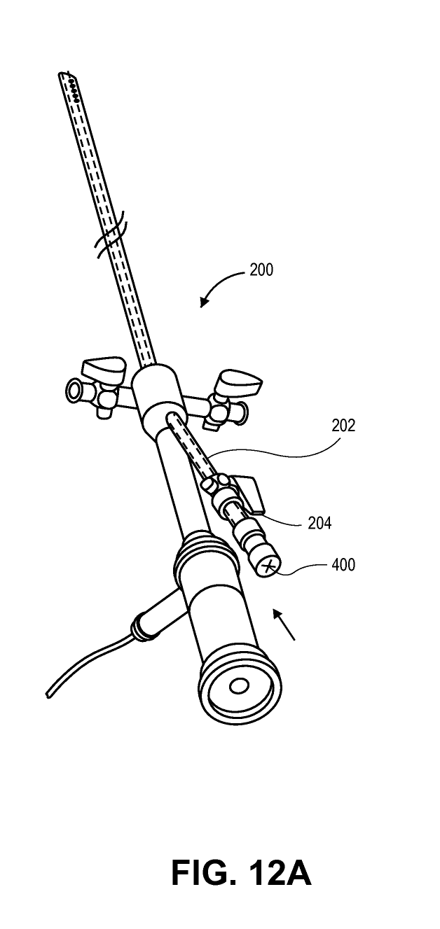

Referring to FIG. 10, a perspective view illustration of a sealant 406 component of a sealing insert in accordance with an embodiment of the invention is shown. In this embodiment, the sealant 406 is shown in a closed configuration. The closed configuration can be characterized by the sealant 406 deforming in one or more directions, resulting in the aperture 426 reducing in profile. As shown, the closed configuration of the sealant 406 can be achieved by rotating a mounting ring 704 near the first end 700 of the sealant relative to a mounting ring 704 near the second end 702 of the sealant such that the sealant twists.

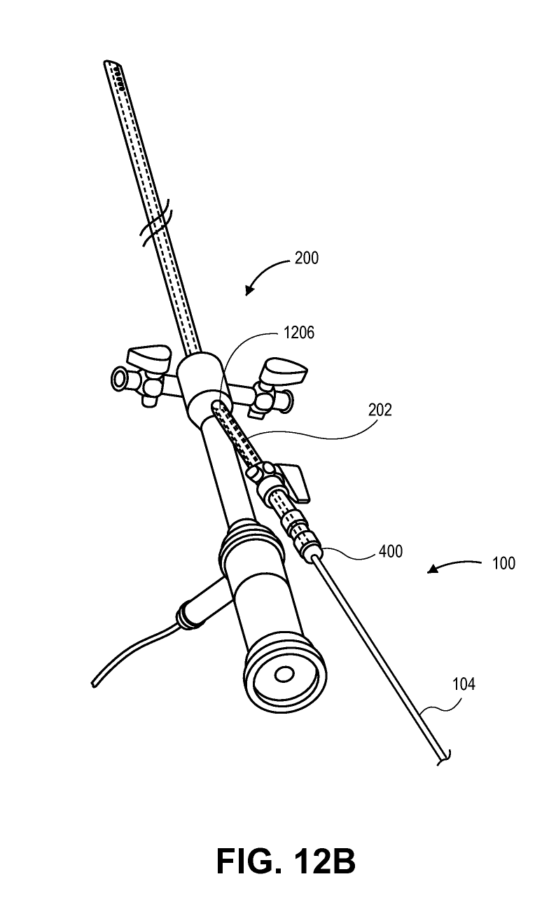

Referring to FIG. 11, a full sectional projected view illustration, taken about section line A′-A′ of FIG. 10, of a sealant 406 component of a sealing insert in accordance with an embodiment of the invention is shown. While in the closed configuration, a cross-section of the sealant 406 can include an aperture 426 that is cinched. The cinching can be caused by the sealant 406 twisting to close like an iris, thereby closing the aperture 426. Thus, a delivery system assembly and/or fluid are prevented from moving freely through the aperture 426 and the sealing insert. Fluid stoppage can be achieved either when a delivery system assembly is inserted through the sealant, or not. That is, the sealant can form a seal against a delivery system assembly by cinching on its surface, or it can form a seal against itself in the closed configuration.

It will be appreciated that alternative manners of achieving a closed aperture 426 can be used, beside the iris-style closure that is described above. For example, rather than rotating the first end 700 and the second end 702 of the sealant 406 relative to each other, the ends can be moved in an axial direction relative to each other. Such relative motion can produce a change in the effective diameter of the aperture 426 by either lengthening or shortening the sealant. Given that in at least one embodiment, the sealant can be formed from an elastomeric material, the axial deformation of the sealant will produce a corresponding change in the cross-sectional area of the sealant, and thus, in the diameter of aperture 426. For example, by moving the first end 700 toward the second end 702, the aperture 426 diameter can be reduced to seal against a delivery system assembly inserted therethrough.

Relative movement of the first end 700 and second end 702 can be achieved by actuating the sealant 406 directly, or by actuating the sealant 406 through other components of the sealing insert 400 assembly. For example, as mentioned above, the cap 404 and the housing 402 can be coupled through a threaded fastener. Thus, rotation of the cap 404 can cause the cap 404 to move both rotationally and axially relative to the housing 402. Given that the sealant 406 can be coupled at either end to the cap 404 and/or housing 402, the relative motion may be imparted to the sealant ends. For example, if the first end 700 of the sealant 406 is frictionally engaged with the housing 402 and the second end 702 of the sealant 406 is frictionally engaged with the cap 404, rotational movement between the cap 404 and housing 402 will also produce a rotational movement between the first end 700 and the second end 702. Similarly, if the first end 700 and second end 702 of the sealant 406 are adhesively bonded to the housing 402 and cap 404 either directly or indirectly through mounting ring 704, then an axial movement between the cap 404 and housing 402 will also produce an axial movement between the first end 700 and the second end 702 of the sealant 406.

Various other features may be incorporated or added to the sealing insert to further enhance the sealing insert functionality. For example, the components of the sealing insert may include surface treatments, such as hydrophilic coatings, in order to provide additional protection against fluid leakage or spray-back. Furthermore, features such as springs may be incorporated to bias the cap, and therefore the aperture, into a given configuration, e.g., a closed configuration. Additionally, other components can be used to isolate the direction of loads within the sealing insert. For example, a follower component (not shown) may be placed between the cap and the sealant to reduce torsion applied to the sealant if the cap is rotated relative to the housing. One skilled in the art would understand that numerous other features and modifications can be made to the sealing insert structure in alternative embodiments that remain within the scope of this description.

As previously mentioned, a delivery system assembly in accordance with embodiments of the invention may be utilized to deliver an insert over a guidewire into an ovarian pathway (e.g. a fallopian tube) of a female body. The sealing insert may protect the tip of the delivery system assembly, guidewire, or insert during insertion into the working channel of a hysteroscope system and reduce the amount of fluid spray-back and leakage associated with inserting the delivery system assembly into the working channel of the hysteroscope system. In an embodiment, the delivery system assembly may include a control device, an elongated catheter sheath having a distal end, and a proximal end connected to the control device. The delivery system assembly can further include an insert that is releasably disposed within the elongated catheter sheath. In an embodiment, the insert extends distally beyond the elongated catheter sheath. In an embodiment, the insert includes a preformed bend.

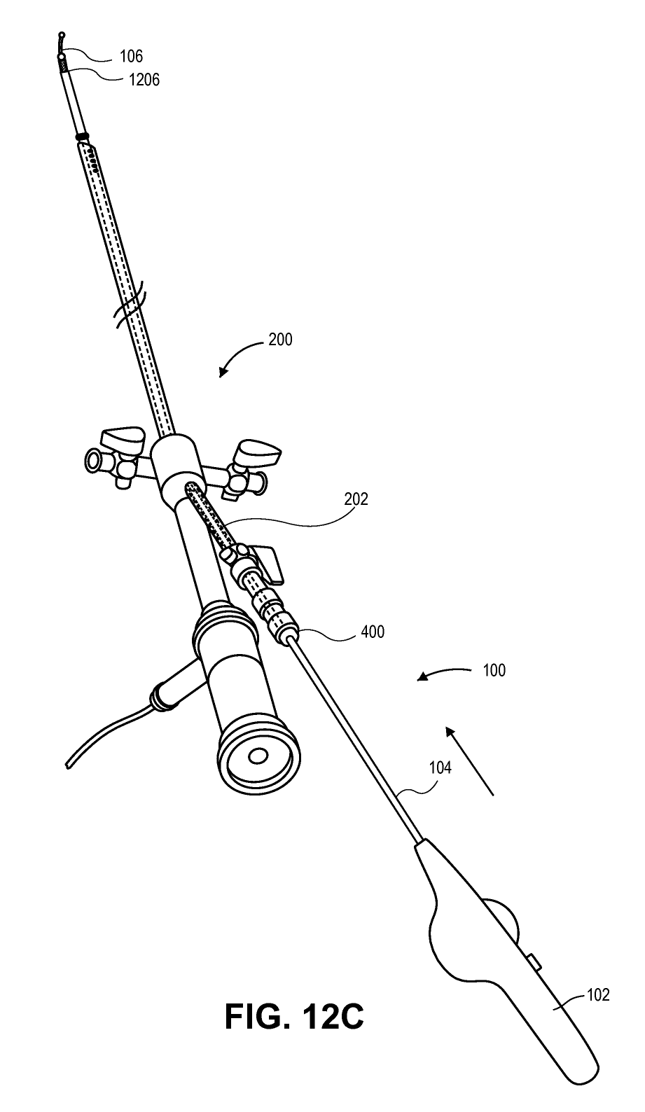

Referring now to FIG. 12A-12C, an isometric view of inserting a delivery system assembly 100 into a working channel 202 of a hysteroscope system 200 in accordance with an embodiment of the invention, is shown. Referring to FIG. 12A, a sealing insert 400 can be inserted into a nozzle of the hysteroscope. That is, the sealing insert 400 can be introduced to seal against a port or working channel 202 of the hysteroscope. An operator can insert the distal end of the delivery system assembly 100 through an access port of the hysteroscope system and into the working channel of the hysteroscope system through the sealing insert 400. In at least one embodiment, the distal end can be inserted directly through the cap port 416, sealant aperture 426, and housing lumen 412, into the working channel 202. In another embodiment, the distal end can be loaded into an introducer either before or after the introducer is advanced through the sealant 406. As mentioned above, the introducer 602 can be an integral component of the sealing insert 400.

Referring now to FIG. 12B, the distal end 1206 of the elongated catheter sheath 104 and insert 106 are inserted into the working channel 202 of the hysteroscope system 200. In an embodiment, the insertion occurs simultaneously with the advancement of an introducer, such as the introducer 602 illustrated in FIG. 6. Simultaneous insertion may avoid fluid spray-back associated with sequentially inserting an introducer 602 followed by an elongated catheter sheath 104.

Referring now to FIG. 12C, the distal end 1206 of the elongated catheter sheath 104 can then be advanced past the hysteroscope system and toward a target location within a body lumen. The cap of sealing insert 400 can be biased to actuate the aperture of the sealant and to control a seal against the elongated catheter sheath 104. Thus, fluid leakage and spray-back is reduced or prevented in advance of device deployment.

The insert 106 can be deployed into the body lumen. Once the insert 106 is deployed into the body lumen, the cap can be biased to open the seal between the sealant and delivery system assembly and the delivery system assembly can be withdrawn from the working channel of the hysteroscope system. In one embodiment, after removal of the delivery system assembly from the working channel of the hysteroscope, the sealant can be moved to a closed configuration to prevent fluid leakage and spray-back. In one embodiment, the sealant closes simultaneously with the withdrawal of the delivery system assembly, e.g., where the sealant is self-closing.

In accordance with embodiments of the invention, multiple components which may reduce or eliminate fluid leakage or spray-back are provided together in a kit. In one embodiment, a kit can include a delivery system assembly, such as one described above, and a sealing insert. In such a kit, the sealing insert can be configured to be inserted into a nozzle of a hysteroscope system. The sealing insert can further include a distal sealing surface and sealant configured to protect against leakage and spray-back and to permit the insertion of the delivery system assembly into a working channel of the hysteroscope. As previously described, the distal sealing surface can include a shape that fits within a variety of nozzles of different outside dimensions to seal against ports or working channels of varying dimensions.

In another embodiment, a kit can include a delivery system assembly, such as the one described above, and a sealing insert having an integrated introducer. The sealing insert can also include a self-closing sealant having, e.g., a slit seal or a duckbill valve. In this manner, the sealant protects against leakage and spray-back when the introducer is not yet inserted. In operation, the distal sealing surface fits into a nozzle housing a working channel of a hysteroscope system. The introducer is then inserted through the self-closing sealant to bias the slit of the sealant toward an open configuration. A delivery system assembly is then advanced through the introducer into the working channel of the hysteroscope.

In the foregoing specification, the invention has been described with reference to specific exemplary embodiments thereof. It will be evident that various modifications may be made thereto without departing from the broader spirit and scope of the invention as set forth in the following claims. The specification and drawings are, accordingly, to be regarded in an illustrative sense rather than a restrictive sense.

What is claimed is:

1. A sealing insert comprising:
a housing comprising a distal sealing surface, a lumen, and a seat, the distal sealing surface being configured to seal against differently sized working channels of a plurality of endoscopes;
a cap comprising a port, the cap coupled with the housing; and
a sealant comprising an aperture, the sealant disposed between the seat and the cap, the aperture aligned with the lumen and the port, whereby a delivery system assembly may be introduced through the port, the aperture, and the lumen into one of the differently sized working channels of the plurality of endoscopes, and wherein the cap is configured to move relative to the housing to actuate the aperture to control a seal between the sealant and the delivery system assembly.

2. The sealing insert of claim 1, wherein the distal sealing surface comprises an outer shape having an outer dimension that varies over a length.

3. The sealing insert of claim 2, wherein the outer shape is selected from the group consisting of frustoconical, stepped, concave, and convex, and wherein the cap actuates the aperture by inwardly deforming the sealant to thereby reduce the cross-sectional size of the aperture.

4. The sealing insert of claim 1, wherein the sealant further comprises a self-closing sealant and wherein the aperture comprises a slit of the self-closing sealant.

5. The sealing insert of claim 4, further comprising an introducer disposed through the port, the introducer comprising a protuberance and a passage, the protuberance disposed between the sealant and the cap, the passage configured to introduce the delivery system assembly through the port, the aperture, and the lumen into one of the differently sized working channels of the plurality of endoscopes.

6. The sealing insert of claim 1, wherein the sealant further comprises a first end and a second end, and wherein movement of the first end relative to the second end actuates the aperture.

7. A sealing insert comprising:
a sealant comprising an aperture;
means for sealing the sealing insert against differently sized working channels of a plurality of endoscopes;
means for actuating the aperture to control a seal between the sealant and a delivery system assembly when the delivery system assembly is introduced through the aperture into one of the differently sized working channels of the plurality of endoscopes.

8. The sealing insert of claim 7, wherein the means for sealing further comprises means for housing at least a portion of the sealant that, and wherein the means for actuating confines the sealant within the means for housing.

9. The sealing insert of claim 7, wherein the means for sealing comprises an outer shape having an outer dimension that varies over a length.

10. The sealing insert of claim 9, wherein the outer shape is selected from the group consisting of frustoconical, stepped, concave, and convex.

11. The sealing insert of claim 7, wherein the sealant further comprises a self-closing sealant and wherein the aperture comprises a slit of the self-closing sealant.

12. The sealing insert of claim 7, further comprising means for introducing the delivery system assembly through the aperture into one of the differently sized working channels of the plurality of endoscopes, the means for introducing comprising a protuberance, wherein the means for actuating the aperture is configured to move the protuberance.

13. The sealing insert of claim 7, wherein the sealant further comprises a first end and a second end, wherein the means for actuating the aperture is configured to move the first end relative to the second end.

14. A kit comprising:
a delivery system assembly comprising:
a control device;
an elongated catheter sheath having a proximal end connected to the control device; and
an insert;
a sealing insert comprising:
a housing comprising a distal sealing surface, a lumen, and a seat, the distal sealing surface being configured to seal against differently sized working channels of a plurality of endoscopes;
a cap comprising a port, the cap coupled with the housing; and
a sealant comprising an aperture, the sealant disposed between the seat and the cap, the aperture aligned with the lumen and the port, whereby the delivery system assembly may be introduced through the port, the aperture, and the lumen into one of the differently sized working channels of the plurality of endoscopes, and wherein the cap is configured to move relative to the housing to actuate the aperture to control a seal between the sealant and the delivery system assembly.

15. The kit of claim 14, wherein the distal sealing surface comprises an outer shape having an outer dimension that varies over a length.

16. The kit of claim 15, wherein the outer shape is selected from the group consisting of frustoconical, stepped, concave, and convex.

17. The kit of claim 14, wherein the sealant further comprises a self-closing sealant and wherein the aperture comprises a slit of the self-closing sealant.

18. The kit of claim 17, further comprising an introducer disposed through the port, the introducer comprising a protuberance and a passage, the protuberance disposed between the sealant and the cap, the passage configured to introduce the delivery system assembly through the port, the aperture, and the lumen into one of the differently sized working channels of the plurality of endoscopes.

19. The kit of claim 14, wherein the sealant further comprises a first end and a second end, and wherein movement of the first end relative to the second end actuates the aperture.
